(19) United States
(12) Patent Application Publication
(10) Pub. No.: US 2025/0114573 A1
(43) Pub. Date: Apr. 10, 2025

(54) CHEMICAL LIQUID INJECTION UNIT

(71) Applicant: Asahi Intecc Co., Ltd., Seto-shi (JP)

(72) Inventors: Kotaro MIZUKAMI, Seto-shi (JP); Toshihiko TSUKAMOTO, Seto-shi (JP)

(73) Assignee: Asahi Intecc Co., Ltd., Seto-shi (JP)

(21) Appl. No.: 18/985,494

(22) Filed: Dec. 18, 2024

Related U.S. Application Data
(63) Continuation of application No. PCT/JP2023/019863, filed on May 29, 2023.

(30) Foreign Application Priority Data
Jun. 24, 2022 (JP) 2022-101967

(51) Int. Cl. A61M 25/06; A61M 25/00; A61M 25/01; A61M 25/04

(52) U.S. Cl. CPC A61M 25/0662; A61M 25/003; A61M 25/0043; A61M 25/0108; A61M 25/04; A61M 2202/0007; A61M 2202/04; A61M 2205/32

(57) ABSTRACT

A chemical liquid injection unit includes a tube. The tube includes a center lumen, at least three puncture catheter ports in communication with the center lumen and open at different positions in a circumferential direction on an outer peripheral surface of the tube, and a support mechanism arranged at a position opposite to each of the at least three puncture catheter ports with respect to an X-axis of the tube and is used to support the tube.

### CROSS-REFERENCE TO RELATED APPLICATIONS

This application is a continuation application of International Application No. PCT Application No. PCT/JP2023/019863 filed May 29, 2023, which claims priority to Japanese Patent Application No. 2022-101967, filed Jun. 24, 2022. The contents of these applications are incorporated herein by reference in their entirety.

### TECHNICAL FIELD

The disclosed embodiments relate to a chemical liquid injection unit.

### BACKGROUND ART

Conventionally, treatments, examinations, and the like have been performed by inserting a catheter-like medical instrument into a tubular organ of a human body such as a blood vessel and a digestive tract. Recently, treatments have also been performed with the use of a catheter-like medical instrument allowing chemical liquid injections by injecting a chemical liquid into body tissue (treatment site). In the treatment with the use of a catheter for chemical liquid injection, the body tissue is punctured with a puncture needle at a catheter distal end that is delivered to the outside of a catheter tube through an opening or a hole provided on the outer peripheral surface of the catheter tube, and a chemical liquid is injected through the puncture needle. For example, Patent Literature 1 discloses a chemical liquid injection apparatus in which predetermined body tissue is punctured with a needle portion of a needle-like tubular body (catheter) projecting through a projection hole provided in one direction of the side of a tubular main body (catheter tube) to inject the chemical liquid into the body tissue through the needle-like tubular body.

### CITATION LIST

### Patent Literature

[Patent Literature 1] Japanese Patent No. 4272905

### SUMMARY

### Technical Problems

In order to inject a chemical liquid into predetermined body tissue that is a treatment site with the use of a catheter tube having a hole in one direction of the side, as shown in Patent Literature 1, the catheter needs to be rotated in a blood vessel in accordance with the position of the body tissue. Moreover, due to the repulsive force generated when puncturing the body tissue with the puncture needle, the catheter tube may be moved in a direction away from the blood vessel, which causes a concern in terms of safety.

In view of the above-described problems, disclosed embodiments are directed to providing a chemical liquid injection unit with improved handling ability and safety.

### Solutions to Problems

In order to achieve the above, a first aspect of the disclosed embodiments provides a chemical liquid injection unit, including a long tube, in which the tube includes a center lumen that extends in an axis direction of the tube and to which a puncture catheter having a puncture needle provided at a distal end is insertable, at least three puncture catheter ports that are in communication with the center lumen and are open at different positions in a circumferential direction on an outer peripheral surface of the tube, and a support mechanism that is arranged at a position opposite to each of the at least three puncture catheter ports with respect to an axis line of the tube and is used to support the tube

In such a disclosed embodiment, a puncture catheter port that is closest to body tissue into which a chemical liquid needs to be injected is selected among the at least three puncture catheter ports, and a puncture catheter is inserted thereinto, which allows injection of the chemical liquid into the body tissue without rotating the catheter in the blood vessel. Moreover, with the support mechanism that is arranged at a position opposite each of the at least three puncture catheter ports, it is possible to suppress the chemical liquid injection unit from moving in a direction away from the blood vessel due to the repulsive force generated when puncturing the body tissue with the puncture needle. That is, in such a disclosed embodiment (disclosed embodiment 1), it is possible to provide a chemical liquid injection unit with improved handling ability and safety.

In the above-described disclosed embodiment (disclosed embodiment 1), the support mechanism may include at least three support wire lumens that extend in the axis direction of the tube and to each of which at least one support wire is insertable, and at least three support wire ports that are in communication respectively with the at least three support wire lumens and are open at different positions in the circumferential direction on the outer peripheral surface of the tube.

In such a disclosed embodiment, one of the support wire ports that is at a position opposite to, with respect to the axis line of the tube, the puncture catheter port through which the puncture catheter is delivered is selected, and the support wire is delivered therethrough, whereby it is possible to suppress the chemical liquid injection unit from moving in a direction away from the blood vessel due to the repulsive force generated when puncturing the body tissue into which the chemical liquid needs to be injected with the puncture needle of the puncture catheter.

In the above-described disclosed embodiments, the at least three puncture catheter ports may be arranged at the same intervals in the circumferential direction on the outer peripheral surface of the tube.

In such a disclosed embodiment, it is possible to appropriately select the puncture catheter port that is closest to the body tissue into which the chemical liquid needs to be injected.

In the above-described disclosed embodiments, the at least three puncture catheter ports may be arranged at different positions in a longitudinal direction on the outer peripheral surface of the tube.

In such a disclosed embodiment, it is possible to select more appropriately the puncture catheter port that is closest to the body tissue into which the chemical liquid needs to be injected.

In the above-described disclosed embodiment, the at least three puncture catheter ports may be arranged at the same intervals in the longitudinal direction on the outer peripheral surface of the tube.

In such a disclosed embodiment, it is possible to select more appropriately the puncture catheter port that is closest to the body tissue into which the chemical liquid needs to be injected.

The above-described disclosed embodiments may include a plurality of puncture catheter sets including the at least three puncture catheter ports, in which the plurality of puncture catheter sets are arranged at different positions in the longitudinal direction of the tube.

In such a disclosed embodiment, it is possible to inject the chemical liquid more easily into a plurality of positions of the body tissue into which the chemical liquid needs to be injected, without moving the chemical liquid injection unit back and forth in the blood vessel.

In the above-described disclosed embodiments, an X-ray impermeable marker may be arranged on at least a part of the outer periphery of each of the at least three puncture catheter ports.

In such a disclosed embodiment, with an X-ray, it is possible to easily confirm the positions of the puncture catheter ports.

In the above-described disclosed embodiment, the support mechanism may include a balloon member having a balloon.

In such a disclosed embodiment, the balloon member at a position opposite to, with respect to the axis line of the tube, the puncture catheter port through which the puncture catheter is delivered is selected as the support mechanism, and the balloon of the balloon member is expanded, whereby it is possible to suppress the chemical liquid injection unit from moving in a direction away from the blood vessel due to the repulsive force generated when puncturing the body tissue into which the chemical liquid needs to be injected with the puncture needle of the puncture catheter.

### Advantageous Effects

In the disclosed embodiments, it is possible to puncture a plurality of sites to be punctured with a puncture needle without rotating a catheter in a blood vessel. Therefore, in the disclosed embodiments, it is possible to provide a chemical liquid injection unit with improved handling ability and safety.

### BRIEF DESCRIPTION OF DRAWINGS

FIG. 1 is a schematic explanatory view illustrating a structure of a chemical liquid injection unit according to one of the disclosed embodiments.

FIG. 2A is a transparent perspective view schematically illustrating a part of a tube of the chemical liquid injection unit of FIG. 1.

FIG. 2B is a diagram illustrating an example in which a puncture catheter and a support wire are inserted into the tube of FIG. 2A, together with a blood vessel.

FIG. 3A is a vertical projection view relative to an axis direction of the tube of FIG. 2A.

FIG. 3B is a vertical projection view relative to an axis direction of the tube of FIG. 2B.

FIG. 4A is a diagram illustrating an example in which X-ray impermeable markers are arranged at puncture catheter ports of the tube of FIG. 2A.

FIG. 4B is a diagram illustrating another example in which X-ray impermeable markers are arranged at the puncture catheter ports of the tube of FIG. 2A.

FIG. 5A is a transparent perspective view schematically illustrating a part of a tube and a part of a distal end portion of a chemical liquid injection unit according to a modification example 1.

FIG. 5B is a diagram illustrating an example in which a guide wire is inserted into the tube of FIG. 5A.

FIG. 6A is a transparent perspective view schematically illustrating a part of a tube of a chemical liquid injection unit according to a modification example 2.

FIG. 6B is a diagram illustrating an example in which a puncture catheter and a support wire are inserted into the tube of FIG. 6A.

FIG. 7A is a vertical projection view relative to an axis direction of the tube of FIG. 6A.

FIG. 7B is a vertical projection view relative to an axis direction of the tube of FIG. 6B.

FIG. 8 is a transparent perspective view schematically illustrating a part of a tube of a chemical liquid injection unit according to a modification example 3.

FIG. 9 is a schematic explanatory view illustrating a structure of a chemical liquid injection unit according to a modification example 4.

FIG. 10 is a transparent perspective view schematically illustrating a part of a tube and a part of a balloon member of the chemical liquid injection unit of FIG. 9.

FIG. 11 is a diagram illustrating a vertical projection view relative to an axis direction of the tube of FIG. 9, together with the balloon member.

### DESCRIPTION OF EMBODIMENTS

Hereinafter, the disclosed embodiments will be described with reference to the drawings. Note that the disclosed embodiments are not limited to ones described in the following, which are merely examples to illustrate the technical features of the disclosed embodiments. Moreover, the shape and size in each drawing is merely illustrated to facilitate understanding of the contents of the disclosed embodiments, and does not precisely reflect the actual shape and size.

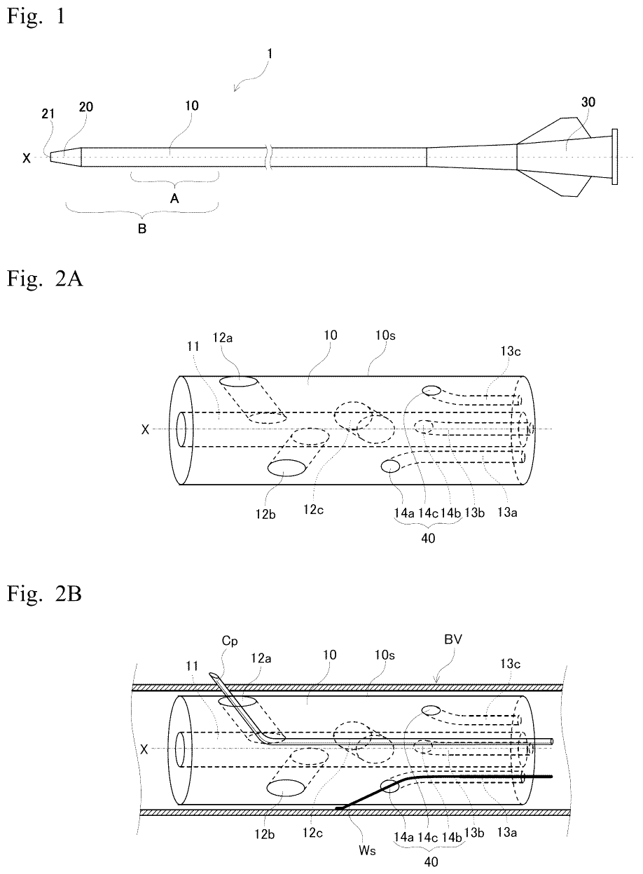

FIG. 1 is a schematic explanatory view illustrating a structure of a chemical liquid injection unit 1 according to one of the disclosed embodiments. The chemical liquid injection unit 1 includes a long tube 10. In particular, the long tube 10 extends further along an axis X-direction than along axes Y-and Z-directions, orthogonal to the axis X-direction. The chemical liquid injection unit 1 is a chemical liquid injection catheter used to inject a chemical liquid into predetermined body tissue that is a treatment site. The chemical liquid injection unit 1 is inserted, for example, into a blood vessel of a heart in which cardiac infarction has occurred so as to inject myocardial cultured cells to a predetermined cardiac muscle.

In the present specification, the “distal end side” is a direction along the axis X direction of the tube 10 forming the chemical liquid injection unit 1, the direction being a direction along which the chemical liquid injection unit 1 is advanced toward predetermined body tissue that is a treatment site. The “proximal end side” indicates a direction along the axis X direction of the tube 10 forming the chemical liquid injection unit 1, the direction being a direction opposite to the above-described distal end side. Moreover, the “distal end” refers to an end on the distal end side of an arbitrary member or portion, and the “proximal end” refers to an end on the proximal end side of an arbitrary member or portion. Note that in FIG. 1, the left side in the drawing is the “distal end side” inserted into a body, and the right side in the drawing is the “proximal end side” operated by a professional.

As illustrated in FIG. 1, the chemical liquid injection unit 1 further includes a distal end portion 20 connected to the distal end of the tube 10, and a branch socket 30 connected to the proximal end of the tube 10.

FIG. 2A is a transparent perspective view schematically illustrating a part of the tube 10 of the chemical liquid injection unit 1 of FIG. 1. FIG. 2A substantially corresponds to a part illustrated with a symbol A in FIG. 1. As illustrated in FIG. 2A, the tube 10 includes a center lumen 11 extending in the axis X direction of the tube 10, at least three puncture catheter ports 12a to 12c that are open at different positions in a circumferential direction on an outer peripheral surface 10s of the tube 10, and a support mechanism 40 that is arranged at a position opposite to each of the at least three puncture catheter ports 12a to 12c with respect to the axis X of the tube 10 and is used to support the tube 10. The center lumen 11 is a lumen into which a puncture catheter Cp having a puncture needle at the distal end thereof is insertable. Each of the puncture catheter ports 12a to 12c is in communication with one common center lumen 11.

In the present specification, the “support mechanism 40 that is arranged at a position opposite to each of the at least three puncture catheter ports 12a to 12c with respect to the axis X of the tube 10” indicates that the support mechanism 40 is arranged at a position opposite to each of the puncture catheters 12a to 12c with respect to the axis X of the tube 10 in the vertical projection view relative to the axis X direction of the tube 10. The “vertical projection view relative to the axis X direction of the tube 10” indicates a projection view of the tube 10 in a plane orthogonal to the axis X of the tube 10.

In the embodiment, the support mechanism 40 includes at least three support wire lumens 13a to 13c extending in the axis X direction of the tube 10, and at least three support wire ports 14a to 14c that are open at different positions in the circumferential direction on the outer peripheral surface 10s of the tube 10. The support wire lumens 13a to 13c are lumens each of which allows at least one support wire Ws to be inserted therethrough. The support wire ports 14a to 14c are respectively in communication with the support wire lumens 13a to 13c.

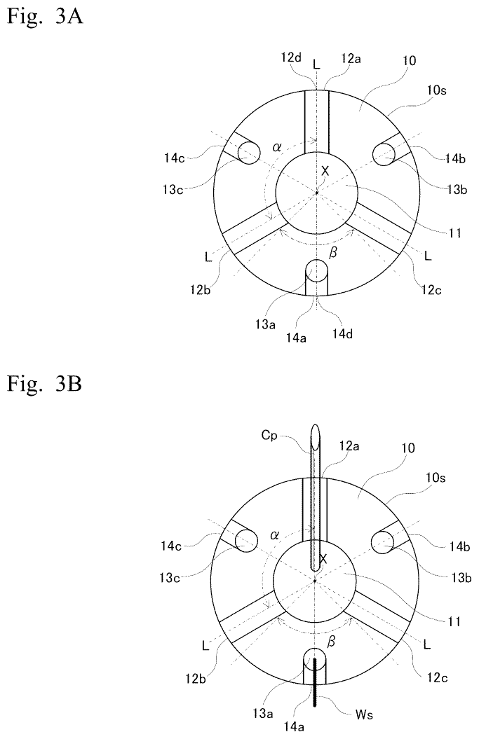

FIG. 3A is a vertical projection view relative to the axis X direction of the tube 10 of FIG. 2A. As illustrated in FIG. 3A, in the vertical projection view relative to the axis X direction of the tube 10, the center lumen 11 is positioned in the center so as to include the axis X of the tube 10. The support wire lumens 13a to 13c are arranged on the outer side of the center lumen 11 so as to surround the center lumen 11.

The inner diameter of the center lumen 11 is configured to be larger than the inner diameters of the support wire lumens 13a to 13c.

As illustrated in FIG. 3A, in the vertical projection view relative to the axis X direction of the tube 10, the puncture catheter ports 12a to 12c and the support wire ports 14a to 14c are arranged at positions opposite to each other with respect to the axis X of the tube 10. Note that the sentence “the puncture catheter ports 12a to 12c and the support wire ports 14a to 14c are arranged at positions opposite to each other with respect to the axis X of the tube 10” indicates that the puncture catheter ports 12a to 12c and the support wire ports 14a to 14c only need to be arranged at positions substantially opposite to each other with respect to the axis X, and does not intend to necessarily limit the arrangement as illustrated in FIG. 3A in which the puncture catheter ports 12a to 12c and the support wire ports 14a to 14c are arranged at positions exactly opposite to each other with respect to the axis X. The centers 14d of the openings of the support wire ports 14a to 14c may be positioned such that an angle β defined by virtual straight lines L passing the centers 12d of the openings of the puncture catheter ports 12a to 12c and the axis X and the axis X is in a range satisfying −45°≤β≤45°.

FIG. 2B is a diagram illustrating an example in which a puncture catheter Cp and a support wire Ws are inserted into the tube 10 of FIG. 2A, together with a blood vessel BV. FIG. 3B is a vertical projection view relative to the axis X direction of the tube 10 of FIG. 2B. Note that the blood vessel BV is omitted in FIG. 3B. As illustrated in FIG. 2B and FIG. 3B, with the chemical liquid injection unit 1, it is possible to select one of the at least three puncture catheter ports 12a to 12c that is closest to the body tissue into which a chemical liquid needs to be injected, and insert the puncture catheter Cp thereinto. The puncture catheter Cp has a puncture needle at the distal end thereof. This allows injection of the chemical liquid into the above-described body tissue without rotating the chemical liquid injection unit 1 in the blood vessel BV.

As illustrated in FIG. 2B and FIG. 3B, in the chemical liquid injection unit 1, one of the support wire ports 14a to 14c that is at a position opposite to, with respect to the axis X of the tube 10, one of the puncture catheter ports 12a to 12c through which the puncture catheter Cp is delivered, may be selected, and the support wire Ws is delivered therethrough to be in contact with the inner wall of the blood vessel BV, whereby it is possible to suppress the chemical liquid injection unit 1 from moving in a direction away from the blood vessel due to the repulsive force generated when puncturing the above-described body tissue with the puncture needle of the puncture catheter Cp.

As the support wire ports 14a to 14c delivering the support wire Ws1, two or more of the at least three support wire ports 14a to 14c may be selected. That is, the support wire Ws may be delivered from each of two or more of the at least three support wire ports 14a to 14c, so as to suppress the chemical liquid injection unit 1 from moving in a direction away from the blood vessel BV. Moreover, two or more support wires Ws may be delivered from one of the at least three support wire ports 14a to 14c, so as to suppress the chemical liquid injection unit 1 from moving in a direction away from the blood vessel BV.

As illustrated in FIG. 3A, the at least three puncture catheter ports 12a to 12c may be arranged at the same intervals in the circumferential direction on the outer peripheral surface 10s of the tube 10. In the example of FIG. 3A, the three puncture catheter ports 12a to 12c are arranged at the same intervals in the circumferential direction on the outer peripheral surface 10s of the tube 10 such that an angle a defined by the straight lines L and the axis X is 120°. In this manner, it is possible to select more appropriately one of the at least three puncture catheters 12a to 12c that is closest to the body tissue into which a chemical liquid needs to be injected.

As illustrated in FIG. 2A, the at least three puncture catheter ports 12a to 12c may be arranged at different positions in a longitudinal direction on the outer peripheral surface 10s of the tube 10. In this manner, it is possible to appropriately select one of the at least three puncture catheter ports 12a to 12c that is closest to the body tissue into which a chemical liquid needs to be injected.

The at least three puncture catheter ports 12a to 12c may be arranged at the same intervals in the longitudinal direction on the outer peripheral surface 10s of the tube 10. In this manner, it is possible to select more appropriately one of the at least three puncture catheter ports 12a to 12c that is closest to the body tissue into which a chemical liquid needs to be injected.

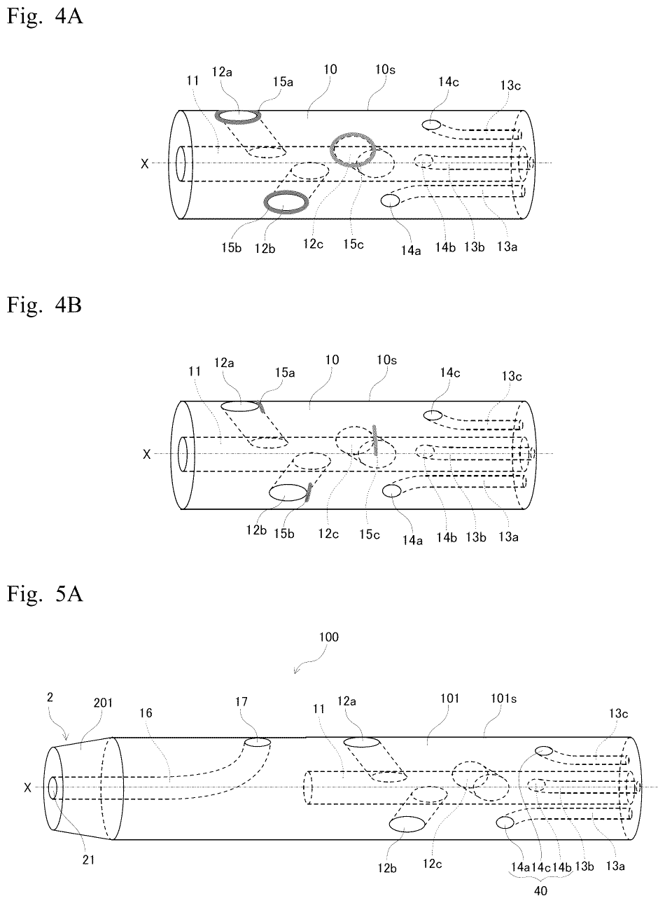

FIG. 4A is a diagram illustrating an example in which an X-ray impermeable marker 15 is arranged at the puncture catheter ports 12a to 12c of the tube 10 of FIG. 2A. FIG. 4B is another diagram illustrating an example in which X-ray impermeable markers 15a to 15c are arranged at the puncture catheter ports 12a to 12c of the tube 10 of FIG. 2A. In the chemical liquid injection unit 1, the X-ray impermeable markers 15a to 15c may be arranged respectively on at least a part of each of the outer peripheries of the at least three puncture catheter ports 12a to 12c. In this manner, with an X-ray, it is possible to easily confirm the positions of the puncture catheter ports 12a to 12c. The X-ray impermeable markers 15a to 15c only need to be arranged such that the positions of the puncture catheter ports 12a to 12c are confirmed with an X-ray, and the arrangement method is not particularly limited. For example, as illustrated in FIG. 4A, the X-ray impermeable markers 15a to 15c may be arranged to surround the outer peripheries of the puncture catheter ports 12a to 12c. Moreover, as illustrated in FIG. 4B, the X-ray impermeable markers 15a to 15c may be arranged linearly in a tangential direction of the outer peripheries of the puncture catheter ports 12a to 12c.

The distal end portion 20 has a distal end opening 21. The distal end opening 21 may be in communication with the center lumen 11. With this configuration, it is possible to guide the guide wire Wg to the distal end opening 21 of the distal end portion 20 through the center lumen 11. That is, in the chemical liquid injection unit 1, the center lumen 11 may function also as a guide wire lumen through which the guide wire Wg is insertable.

The branch socket 30 is configured to allow attachment of three connectors. For example, the first and second connectors may be in communication with the center lumen 11 to correspond to the center lumen 11, and the third connector may be in communication with the support wire lumens 13a to 13c. In this case, it is possible, for example, to guide the puncture catheter Cp inserted from the opening portion of the first connector to one of the puncture catheter ports 12a to 12c through the center lumen 11, and guide the guide wire Wg inserted from the opening portion of the second connector to the distal end opening 21 through the center lumen 11. Further, the support wire Ws inserted from the opening portion of the third connector can be guided to one of the support wire ports 14a to 14c through one of the support wire lumens 13a to 13c.

### MODIFICATION EXAMPLE 1

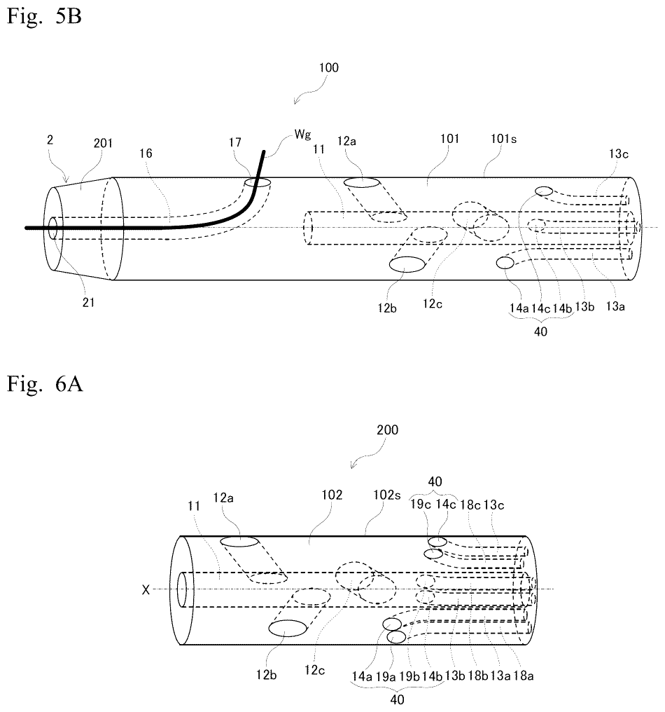

FIG. 5A is a transparent perspective view schematically illustrating a part of a tube 101 and a part of a distal end portion 201 of a chemical liquid injection unit 100 according to a modification example 1. FIG. 5A substantially corresponds to a part illustrated with a symbol B in FIG. 1. FIG. 5B is a diagram illustrating an example in which the guide wire Wg is inserted into the tube 101 of FIG. 5A. As illustrated in FIG. 5A and FIG. 5B, the chemical liquid injection unit 100 may include a guide wire lumen 16 through which the guide wire Wg is insertable, separately from the center lumen 11. The guide wire lumen 16 extends in the axis direction of the tube 101, and communicates with the distal end opening 21 of the distal end portion 201. As illustrated in FIG. 5A and FIG. 5B, a guide wire port 17 in communication with the guide wire lumen 16 may be arranged on an outer peripheral surface 101s of the tube 101. A plurality of guide wire ports 17 may be present at different positions in the circumferential direction of the tube 101. Each of the plurality of guide wire ports 17 may be in communication with one common center lumen 11.

Regarding the three connectors attachable to the branch socket 30, in the modification example 1, the first connector may be in communication with the center lumen 11 to correspond to the center lumen 11, the second connector may be in communication with the guide wire lumen 16 to correspond to the guide wire lumen 16, and the third connector may be in communication with the support wire lumens 13a to 13c, for example. In this case, it is possible, for example, to guide the guide wire Ws inserted from the opening portion of the second connector to the distal end opening 21 through the guide wire lumen 16.

In the modification example 1, the inner diameter of the guide wire lumen 16 is configured to be larger than the inner diameters of the support wire lumens 13a to 13c. The inner diameter of the guide wire lumen 16 and the inner diameter of the center lumen 11 may be the same or different. The inner diameter of the guide wire lumen 16 may be configured to be smaller than the inner diameter of the center lumen 11.

### MODIFICATION EXAMPLE 2

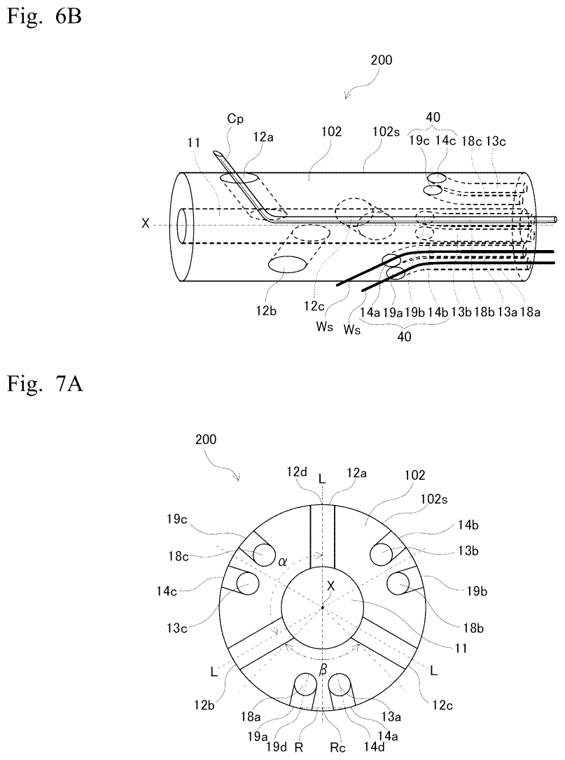

FIG. 6A is a transparent perspective view schematically illustrating a part of a tube 102 of a chemical liquid injection unit 200 according to a modification example 2. In the chemical liquid injection unit 200, the support mechanism 40 includes at least three first support wire lumens 13a to 13c extending in the axis X direction of the tube 102, at least three first support wire ports 14a to 14c open at different positions in the circumferential direction on an outer peripheral surface 102s of the tube 102, at least three second support wire lumens 18a to 18c extending in the axis X direction of the tube 102, and at least three second support wire ports 19a to 19c open at different positions in the circumferential direction on the outer peripheral surface 102s of the tube 102. The first support wire lumens 13a to 13c correspond to the above-described support wire lumens 13a to 13c, respectively. The first support wire ports 14a to 14c correspond to the above-described support wire ports 14a to 14c, respectively. The second support wire lumens 18a to 18c are lumens each of which allows at least one support wire Ws to be inserted therethrough. The second support wire ports 19a to 19c are respectively in communication with the second support wire lumens 18a to 18c.

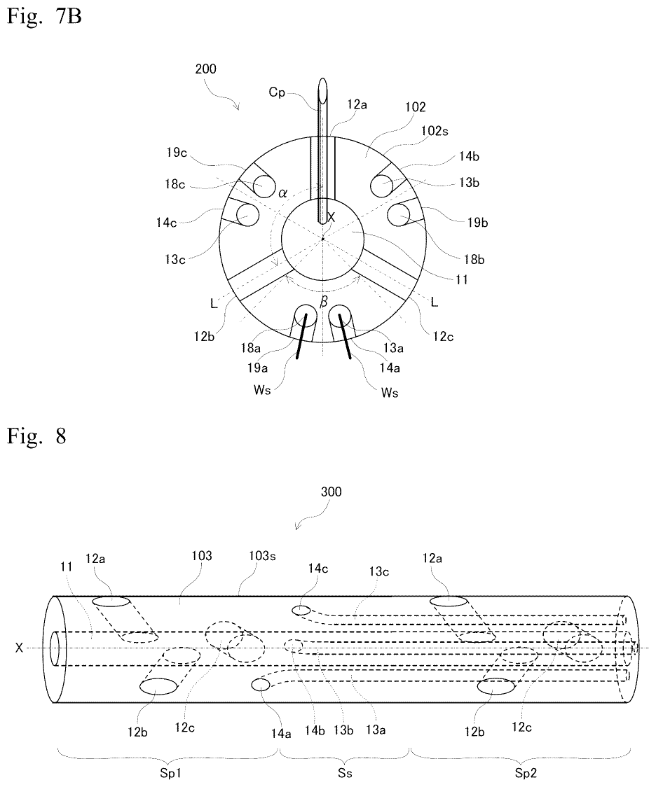

FIG. 7A is a vertical projection view relative to the axis X direction of the tube 102 of FIG. 6A. In the example illustrated in FIG. 7A, in the vertical projection view relative to the axis X direction of the tube 102, the second support wire lumens 18a to 18c are arranged at positions different from the first support wire lumens 13a to 13c in the axis X direction, that is, at positions not overlapping the first support wire lumens 13a to 13c in the axis X direction. The second support wire lumens 18a to 18c are arranged on the outer side of the center lumen 11 to surround the center lumen 11.

The inner diameter of the center lumen 11 is configured to be larger than the inner diameters of the second support wire lumens 18a to 18c. The inner diameters of the second support wire lumens 18a to 18c and the inner diameters of the first support wire lumens 13a to 13c may be the same or different.

As illustrated in FIG. 7A, in the vertical projection view relative to the axis X direction of the tube 102, the puncture catheter ports 12a to 12c and the second support wire ports 19a to 19c are arranged at positions opposite to each other with respect to the axis X of the tube 102. Note that the sentence “the puncture catheter ports 12a to 12c and the second support wire ports 19a to 19c are arranged at positions opposite to each other with respect to the axis X of the tube 102” indicates that the puncture catheter ports 12a to 12c and the second support wire ports 19a to 19c only need to be arranged at positions substantially opposite to each other with respect to the axis X, and does not intend to necessarily limit the arrangement in which the puncture catheter ports 12a to 12c and the second support wire ports 19a to 19c are arranged at positions exactly opposite to each other with respect to the axis X. The centers 19d of the openings of the second support wire ports 19a to 19c may be positioned such that an angle β defined by the virtual straight lines L and the axis X is in a range satisfying −45°≤β≤45°. Moreover, as illustrated in FIG. 7A, regarding the first support wire ports 14a to 14c and the second support wire ports 19a to 19c that are closest to each other in the axis X direction may be arranged such that middle points Rc of virtual straight lines R connecting the centers 14d of the openings of the first support wire ports 14a to 14c and the centers 19d of the openings of the second support wire ports 19a to 19c are at positions opposite to the puncture catheter ports 12a to 12c with respect to the axis X of the tube 102. The definition of the phrase “arranged at a position opposite to . . . with respect to the axis X of the tube 102” is as described above. That is, the middle point Rc may also be positioned such that the angle β defined by the virtual lines L and the axis X is in a range satisfying −45°≤β≤45°.

FIG. 6B is a diagram illustrating an example in which the puncture catheter Cp and the support wire Ws are inserted into the tube 102 of FIG. 6A. FIG. 7B is a vertical projection view relative to the axis X direction of the tube 102 of FIG. 6B. As illustrated in FIG. 6B and FIG. 7B, in the chemical liquid injection unit 200 of the modification example 2, one of the first support wire ports 14a to 14c and one of the second support wire ports 19a to 19c that are at positions opposite to, with respect to the axis X of the tube 10, one of the puncture catheter ports 12a to 12c through which the puncture catheter Cp is delivered, may be selected, and the support wires Ws are delivered therethrough to be in contact with the inner wall of the blood vessel BV, whereby it is possible to suppress the chemical liquid injection unit 200 from moving in a direction away from the blood vessel BV due to the repulsive force generated when puncturing the predetermined body tissue that is a treatment site with the puncture needle of the puncture catheter Cp.

As the second support wire ports 19a to 19c delivering the support wire Ws, two or more of the at least three second support wire ports 19a to 19c may be selected. That is, the support wire Ws is delivered from each of two or more of the at least three second support wire ports 19a to 19c, so as to suppress the chemical liquid injection unit 200 from moving in a direction away from the blood vessel BV. Moreover, two or more support wires Ws may be delivered from one of the at least three second support wire ports 19a to 19c, so as to suppress the chemical liquid injection unit 200 from moving in a direction away from the blood vessel BV.

### MODIFICATION EXAMPLE 3

FIG. 8 is a transparent perspective view schematically illustrating a part of a tube 103 of a chemical liquid injection unit 300 according to a modification example 3. The chemical liquid injection unit 300 of the modification example 3 includes a plurality of puncture catheter sets Sp (Sp1, Sp2) including at least three puncture catheter ports 12a to 12c, and has the same configuration as the chemical liquid injection unit 1 including the tube 10, except for the plurality of puncture catheter sets Sp arranged at different positions in the longitudinal direction of the tube 103.

The chemical liquid injection unit 300 includes the plurality of puncture catheter sets Sp (Sp1, Sp2) arranged at different positions in the longitudinal direction of the tube 103. Thus, the number of choices of the puncture catheter ports 12a to 12c delivering the puncture catheter Cp is increased, which facilitates injection of a chemical liquid into a plurality of positions of predetermined body tissue that is a treatment site without moving the chemical liquid injection unit 300 back and forth in the blood vessel BV.

Here, a set including at least three support wire ports 14a to 14c is defined as a support set Ss. As illustrated in FIG. 8, in the chemical liquid injection unit 300 of the modification example 3, the support set Ss may be arranged between the puncture catheter set Sp1 and the puncture catheter set Sp2 in the axis X direction. With such a configuration, the support set Ss suppresses the chemical liquid injection unit 300 from moving in a direction away from the blood vessel BV in both of the case in which the puncture wire ports 12a to 12c of the puncture catheter set Sp1 on the distal end side is used and the case in which the puncture wire ports 12a to 12c of the puncture catheter set Sp2 on the proximal end side is used.

The chemical liquid injection unit 300 may include a plurality of support sets Ss. For example, the puncture catheter set Sp, the support set Ss, the puncture catheter set Sp, the support set Ss, . . . may be arranged in this order from the distal end side toward the proximal end side. In a case where the plurality of support sets Ss are provided, the support wire ports 14a to 14c of the plurality of support sets Ss may be respectively in communication with the common support wire lumens 13a to 13c.

The form of the modification example 2 is applicable to the chemical liquid injection unit 300 of the modification example 3. That is, the chemical liquid injection unit 300 may include the second support set Ss2 including the at least three second support wire ports 19a to 19c in addition to the first support set Ss1 including the at least three first support wire ports 14a to 14c. The chemical liquid injection unit 300 may include a plurality of the second support sets Ss2.

### MODIFICATION EXAMPLE 4

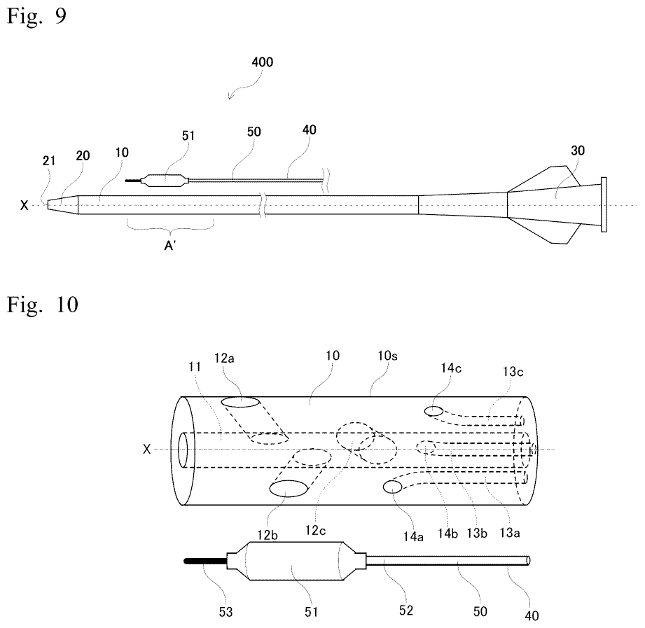

FIG. 9 is a schematic explanatory view illustrating a structure of a chemical liquid injection unit 400 according to a modification example 4. In the chemical liquid injection unit 400, the support mechanism 40 includes a balloon member 50 having a balloon 51. That is, the chemical liquid injection unit 400 of the modification example 4 has the same configuration as the chemical liquid injection unit 1, except for the puncture catheter ports 12a to 12c arranged at positions opposite to the balloon members 50 with respect to the axis X of the tube 10.

FIG. 10 is a transparent perspective view schematically illustrating a part of the tube 10 and a part of the balloon member 50 of the chemical liquid injection unit 400. FIG. 10 substantially corresponds to a part illustrated with a symbol A′ in FIG. 9. The balloon member 50 further includes a long tube 52 and a distal tip 53 attached on the distal end side of the tube 52, in addition to the balloon 51. The balloon 51 is provided between the distal tip 53 and the tube 52. The balloon member 50 may be integrated with the tube 10 on the proximal end side, or may be a body separate from the tube 10. In a case where the balloon member 50 is a body separate from the tube 10, the balloon member 50 may further include a connector connected to the proximal end of the tube 52.

The balloon member 50 can supply fluid to the inner space of the balloon 51 through the tube 52. Consequently, the balloon 5 is expanded.

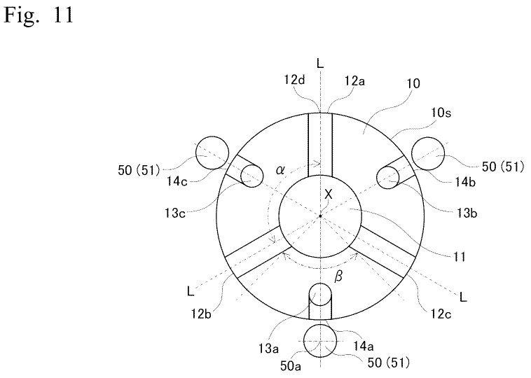

FIG. 11 is a diagram illustrating the vertical projection view relative to the axis X direction of the tube 10 of FIG. 9, together with the balloon members 50. As illustrated in

FIG. 11, in the vertical projection view relative to the axis X direction of the tube 10, the puncture catheter ports 12a to 12c are arranged at positions opposite to the balloon members 50 with respect to the axis X of the tube 10. Note that the sentence “the puncture catheter ports 12a to 12c are arranged at positions opposite to the balloon members 50 with respect to the axis X of the tube 10” indicates that the puncture catheter ports 12a to 12c only need to be arranged at positions substantially opposite to the balloon members 50 with respect to the axis X, and does not intend to necessarily limit the arrangement as illustrated in FIG. 11 in which the puncture catheter ports 12a to 12c are arranged at positions exactly opposite to the balloon members 50 with respect to the axis X. That is, a center 50a of the balloon member 50 may also be positioned such that the angle β defined by the virtual lines L and the axis X is in a range satisfying −45°≤β≤45°.

In the chemical liquid injection unit 400, the support mechanism 40 includes the balloon member 50 having the balloon 51, which suppresses the chemical liquid injection unit 400 from moving in a direction away from the blood vessel BV due to the repulsive force generated when puncturing the predetermined body tissue that is a treatment site with the puncture needle.

As the balloon member 50, two or more balloon members 50 may be used. That is, two or more balloons 51 may function as the support mechanism 40. In this case, the stability of the chemical liquid injection unit 400 is further improved. FIG. 11 illustrates an example in which three balloon members 50 are used as the balloon member 50.

The above-described center lumen 11, support wire lumens (first support wire lumens) 13a to 13c, second support wire lumens 18a to 18c, and guide wire lumen 16 are tube bodies formed of resin. Resin materials for forming the tube bodies of these lumens are not particularly limited, but resins having excellent slidability with other members are preferable. Examples thereof include fluororesin such as PTFE (polytetrafluoroethylene), PVDF (polyvinylidene fluoride), PFA (perfluoroalkoxy alkane), FEP (perfluoroethylene propene), and ETFE (ethylene tetrafluoroethylene), PE (polyethylene), and PP (polypropylene). Note that the tube bodies forming the center lumen 11, the support wire lumens (first support wire lumens) 13a to 13c, the second support wire lumens 18a to 18c, and the guide wire lumen 16 may be formed of the same resin material or formed of mutually different resin materials.

A reinforcing body may be arranged on the outer periphery of the above-described tube body. The above-described reinforcing body is, for example, a braided body (metal braid layer) formed by braiding a plurality of wires in a net-like shape (mesh shape). The above-described reinforcing body may cover the entire outer periphery of the above-described tube body or cover a part of the periphery of the above-described tube body. Moreover, a resin outer layer tube with the above-described reinforcing body embedded therein may be arranged on the outer periphery of the above-described tube body.

The tubes 10, 101, 102, and 103 are formed of resin. For example, in the tube 10, the tube body forming the center lumen 11 and the tube bodies forming the support wire lumens (first support wire lumens) 13a to 13c are covered. The resin material for forming the tubes 10, 101, 102, and 103 is not particularly limited, and examples thereof include polyamide, polyamide elastomer, polyester, polyurethane, and polyurethane elastomer. The tubes 10, 101, 102, and 103 may be formed of a single resin material, or may be divided into a plurality of regions formed of a plurality of resin materials having different characteristics. Moreover, the resin for forming the tubes 10, 101, 102, and 103 may contain tungsten powder, and the hardness of the resin may be changed depending on the content thereof. Containing tungsten powder that is X-ray impermeable powder in the resin for forming the tubes 10, 101, 102, and 103 enables a professional such as a doctor to accurately grasp the position of the chemical liquid injection unit 1 at the time of treatment.

The above has described the chemical liquid injection unit according to the disclosed embodiments with reference to the drawings. However, the disclosed embodiments are not limited to the above-described ones, and various changes can be made. For example, in the above-described embodiments, the chemical liquid injection unit is described as a catheter used to inject a chemical liquid into predetermined body tissue that is a treatment site, but the catheter to which the chemical liquid injection unit of the disclosed embodiments is applied is not limited to a use for chemical liquid injection, but may be applied to an endoscope or the like, for example.

### REFERENCE SIGNS LIST

1, 100, 200, 300, 400 chemical liquid injection unit
10, 101, 102, 103 tube
11 center lumen
12a to 12c puncture catheter port
13a to 13c support wire lumen (first support wire lumen)
14a to 14c support wire port (first support wire port)
15a to 15c X-ray impermeable marker
16 guide wire lumen
17 guide wire port
18a to 18c second support wire lumen
19a to 19c second support wire port
20, 201 distal end portion
21 distal end opening
30 branch socket
40 support mechanism
50 balloon member
51 balloon
52 tube
53 distal tip
Sp puncture catheter set
Ss support set
Cp puncture catheter
Ws support wire
Wg guide wire

What is claimed is:

1. A chemical liquid injection unit, comprising:
a tube, wherein
the tube includes
a center lumen that extends in an axial direction of the tube and to which a puncture catheter having a puncture needle provided at a distal end is insertable,
at least three puncture catheter ports that are in communication with the center lumen and are open at different positions in a circumferential direction on an outer peripheral surface of the tube, and
a support mechanism opposite each of the at least three puncture catheter ports with respect to an axis line of the tube and is used to support the tube.

2. The chemical liquid injection unit according to claim 1, wherein
the support mechanism includes
at least three support wire lumens that extend in the axis direction of the tube and to each of which at least one support wire is insertable, and
at least three support wire ports that are in communication_respectively with the at least three support wire lumens and are open at different positions in the circumferential direction on the outer peripheral surface of the tube.

3. The chemical liquid injection unit according to claim 1, wherein the at least three puncture catheter ports are arranged at same intervals in the circumferential direction on the outer peripheral surface of the tube.

4. The chemical liquid injection unit according to claim 1, wherein the at least three puncture catheter ports are arranged at different positions in a longitudinal direction on the outer peripheral surface of the tube.

5. The chemical liquid injection unit according to claim 4, wherein the at least three puncture catheter ports are arranged at same intervals in the longitudinal direction on the outer peripheral surface of the tube.

6. The chemical liquid injection unit according to claim 1, further comprising a plurality of puncture catheter sets including the at least three puncture catheter ports, wherein
the plurality of puncture catheter sets are arranged at different positions in a longitudinal direction of the tube.

7. The chemical liquid injection unit according to claim 1, wherein an X-ray impermeable marker is arranged on at least a part of an outer periphery of each of the at least three puncture catheter ports.

8. The chemical liquid injection unit according to claim 1, wherein the support mechanism includes a balloon member having a balloon.

9. The chemical liquid injection unit according to claim 1, further comprising a guide wire lumen to which a guide wire is to be inserted.

10. The chemical liquid injection unit according to claim 9, wherein the center lumen serves as the guide wire lumen.

11. The chemical liquid injection unit according to claim 9, wherein the guide wire lumen is separate from the center lumen.

12. The chemical liquid injection unit according to claim 11, wherein the guide wire lumen has a different diameter than the center lumen.

13. The chemical liquid injection unit according to claim 1, the support mechanism includes
at least three pairs of support wire lumens that extend in the axis direction of the tube and to each of which at least one support wire is insertable, and
at least three pairs of support wire ports that are in communication respectively with the at least three pairs of support wire lumens and are open at different positions in the circumferential direction on the outer peripheral surface of the tube.

14. The chemical liquid injection unit according to claim 13, wherein each pair of support wire ports are arranged such that middle points of a virtual straight line connecting centers of the openings of the pair are at positions opposite to the puncture catheter ports.
